(19) United States
(12) Patent Application Publication
(10) Pub. No.: US 2006/0134738 A1
(43) Pub. Date: Jun. 22, 2006

(54) Gene expression inducing fusion protein and method for controlling gene expression Induction

(76) Inventors: Christel Boutonnet, Albi (FR); Stephan Vagner, Clermont Le Fort (FR); Jean-Charles Faye, Montesquieu-Volvestre (FR); Gilles Favre, Cugnaux (FR); Abdelhakim Kharrat, Montgiscard (FR); Khalil Bouayadi, Toulouse (FR); Laurence Vercaemer, Paris (FR)

Correspondence Address: PATZIK, FRANK & SAMOTNY LTD., 150 SOUTH WACKER DRIVE, SUITE 900, CHICAGO, IL, 60606, US

(21) Appl. No.: 10/542,471

(22) PCT Filed: Jan. 15, 2004
(86) PCT No.: PCT/FR04/00073

§ 371 Date: Jul. 15, 2005

(30) Foreign Application Priority Data
Jan. 15, 2003 (FR) 03/00422

(51) Int. Cl. C07K 14/47; C07H 21/04; C12P 21/06; C12N 5/08

(52) U.S. Cl. 435/69.1; 435/320.1; 435/325; 530/350; 536/23.2; 435/367

(57) ABSTRACT

The invention relates to a gene expression inducing fusion protein comprising a) a ribonucleic acid linking peptide domain and a post-transcriptional gene expression activator domain and b) a domain enabling cytoplasmic membrane delocalization. The invention also relates to a modulatable permanent external method for controlling gene expression induction by modulating the state of post-translational modulation of the gene expression inducing fusion protein for the expression of said gene.

The invention relates to a method for controlling post-transcriptional gene expression induction, and in particular to a method for controlling gene translation induction.

In the field of hereditary or genetic disease treatment, gene therapy-type treatments, which make it possible to replace or correct defective genes, are in the process of being developed. Gene therapy can also have important applications in the delivery of proteins, for example cytokines, anti-oncogenes, hormones or antibodies, said proteins having a therapeutic activity in the treatment of cancer-type pathologies or viral infections.

However, even though it is possible to induce the expression of a gene, and therefore of the protein encoded by the gene, the lack of means for controlling the expression of this protein is a barrier to the use of such techniques, in particular in the context of a gene therapy. A simple and safe means for controlling the induction of expression of a gene, and therefore of the protein that it encodes, would find applications both in gene therapy protocols and in the establishment of cell lines expressing a transgene, or alternatively in the production of transgenic animals.

Means for gene expression induction are already known.

However, most of the means described are based on transcriptional inductions and have major drawbacks for use in gene therapy. Mention will in particular be made of the article by Mills (2001) Changing colors in mice: an inducible system that delivers, GENES & DEVELOPMENT, 15:1461-1467. The activating chimeric proteins of these various systems of the prior art cause immunogenic reactions and/or the pharmacological inducers used (tetracycline, rapamycin) bring about undesired cellular and tissue reactions.

Mention will also be made of document GB 2 273 708, which describes mainly a method for screening compounds that modulate the protein/cell membrane association, said method using a heterologous protein that includes a recognition sequence for adhesion to the cell membrane and a transcription activator. The entire description refers to transcription activation. Moreover, reference is made to the possibilities of activating or inducing (“switching on”) a gene by means of the fusion protein, but the control as such, i.e. the mastering, of the expression is never discussed or suggested.

Patent application WO 99/11801 also describes the activation of gene transcription using a transcription factor released from the cell membrane by a protease. The method of action is, in this case, proteolytic cleavage. In addition, it is once again the activation which is sought and discussed, and not gene expression control.

Moreover, patent application WO 00/53779 describes a method for the translational induction of gene expression, using a ribosome recruitment protein, i.e. a protein similar to eIF4G or a translationally active derivative or fragment thereof. This protein is fused with a protein, or a protein fragment or derivative, capable of binding to a heterologous protein-binding site or HBS present on the mRNA. An example of an mRNA-binding protein is IRP-1. An example of an HBS is the IRE (Iron Responsive Element). The fusion protein obtained can bind to an HBS, and plays the role of an activator of translation of the mRNA, and therefore of the expression of the protein(s) encoded by this mRNA downstream of the HBS.

That document also explains that the expression of the protein(s) can be controlled. This control is carried out using various HBSs on the mRNA. It is therefore a control, a priori, exerted at the beginning of the treatment or of the experiment, rather than a real mastering of induction. Once the number and the nature of the HBSs has been chosen, the translation induction can no longer be modulated.

Consequently, there is still a real need for a means of modulatable control of post-transcriptional gene expression induction, which control should be capable of being exerted at any moment during the implementation of the post-transcriptional expression induction, both qualitatively and quantitatively.

The subject of the present invention is therefore a modulatable permanent external method for controlling post-transcriptional gene expression induction, said control making it possible to initiate, stop and modulate the induction at any moment. The expression “post-transcriptional expression induction” is intended to mean the induction of any post-transcriptional step of gene expression, i.e. in particular translation, pre-mRNA splicing, pre-mRNA polyadenylation, etc., and in particular translation.

In the context of the invention, the post-transcriptional gene expression induction is carried out by expressing a specific fusion protein, called inducer fusion protein.

The inducer fusion protein used to induce the post-transcriptional gene expression according to the invention comprises, firstly, a ribonucleic acid-binding peptide domain and a domain for activating the expression of said gene and, secondly, a domain enabling cell membrane delocalization.

Any protein or polypeptide known, to those skilled in the art, to induce gene expression can be used as an inducer fusion protein according to the invention, either as it is, if it comprises a membrane delocalization domain in addition to the gene expression-activating domain, or as a fusion protein or polypeptide in combination with a membrane delocalization domain. In particular, any fusion protein known, to those skilled in the art, to induce translation can be used, and in particular those described in patent application WO 00/53779 mentioned above, namely any fusion protein consisting of a protein similar to eIF4G or to a translationally active derivative or fragment thereof, fused with an mRNA-binding protein or protein fragment or derivative. Mention will be made, without implied limitation, of the fusion protein consisting of eIF4G1 fused with IRP-1.

The inducer fusion protein according to the invention therefore comprises a membrane delocalization domain. This domain is intrinsically present or is fused with a ribonucleic acid-binding peptide domain and an expression-activating domain.

A membrane delocalization domain is a peptide domain which is the site of a post-translational modification of the protein that allows delocalization, to the cell membranes (cytoplasmic, nuclear, mitochondrial membranes, etc.), of the protein comprising said domain. In particular, the post-translational modification may be the modification of a cysteine, in the carboxy-terminal CAAX domain of the protein of interest, using enzymes of farnesyltransferase or geranyl-geranyl transferase type. CAAX signifies “Cysteine-Aliphatic amino acid-Aliphatic amino acid-amino acid X”, where X may be a methionine, a glutamine, a serine or a threonine. The post-translational modification may, by way of nonlimiting examples, be farnesylation, geranylgeranylation, myristylation or palmytoylation, or any other modification known to those skilled in the art. When the protein has the peptide sequence resulting from the post-translational modification, it is targeted to the membrane.

The use of farnesylation has already been envisioned in anticancer treatments in order to render inactive certain proteins (small G proteins, etc.) involved in pathologies, but it has never been used in the context of gene expression induction.

The method for controlling post-transcriptional gene expression induction according to the invention comprises the modulatable permanent external control of gene expression induction by modulation of the state of post-translational modification of the fusion protein that induces the post-transcriptional expression of the gene, and more particularly of the membrane delocalization domain. This control is carried out by using an appropriate inhibitor of said post-translational modification. If the post-translational modification of the fusion protein is farnesylation, an appropriate inhibitor is a farnesyltransferase inhibitor, such as FTI 277. If the post-translational modification of the fusion protein is geranylgeranylation, an appropriate inhibitor is a geranyltransferase inhibitor, such as GGTI 298. Those skilled in the art will be able to determine the appropriate inhibitor, given the post-translational modification domain present.

In the absence of inhibitor, the post-translational modification of the inducer fusion protein will take place. The fusion protein, once synthesized and equipped with the peptide sequence for inducing post-translational modification, will be anchored in a cell membrane, and will not therefore be able to play its gene expression-activating role.

Conversely, in the presence of an appropriate inhibitor, the post-translational modification will not take place, and the inducer fusion protein will no longer be targeted to the membrane and will therefore be able to play its gene expression-activating role.

A subject of the invention is a gene expression-inducing fusion protein comprising, firstly, a ribonucleic acid-binding peptide domain and a domain for activating the post-transcription expression of said gene and, secondly, a domain enabling delocalization to the cytoplasmic membrane.

By way of example of an inducer fusion protein according to the invention, mention will be made of the R17-4G-CVLS fusion proteins, where CVLS corresponds to a “CAAX” domain described as being responsible for the delocalization of the protein to the membrane.

A subject of the invention is also a nucleic acid comprising a sequence encoding the expression-inducing fusion protein according to the invention. This nucleic acid may be a double-stranded or single-stranded DNA, a cDNA, or an RNA such as an mRNA.

A subject of the invention is also an expression vector, in particular a plasmid, comprising a nucleic acid according to the invention.

A subject of the invention is also a recombined cell comprising a nucleic acid according to the invention, integrated into its genome, and also a cell line comprising such a nucleic acid. By way of nonlimiting examples, mention will be made of SHKep-1 human hepatoma cells, HeLa cells or CHO cells.

A subject of the invention is also a nonhuman transgenic organism, for example a mouse, comprising a nucleic acid according to the invention as a transgene.

More specifically, according to the invention, the modulatable permanent external method for controlling post-transcriptional gene expression induction in a recombined cell or in a nonhuman transgenic organism, comprising a nucleic acid comprising a sequence encoding an inducer fusion protein according to the invention, or comprising a vector comprising said nucleic acid, involves modulating the state of post-translational modification of the gene expression-inducing fusion protein using an appropriate inhibitor of the post-translational modification of said fusion protein.

The control of the post-transcriptional gene expression induction is therefore simple, modulatable and permanent: it is possible, at any time, to commence, stop or recommence the addition of the appropriate post-translational modification inhibitor, or to modify the amounts added, in order to control the induction both qualitatively and quantitatively. Moreover, given the high degrees of induction that can be obtained, it is possible to use the fusion protein inhibitor at a low concentration, thus avoiding any possible toxicity problems.

One of the advantages of the present invention is the possibility of making a cell express a reporter gene and an effector gene, the reporter gene comprising a binding site for a polypeptide and at least one gene of interest, and the effector gene encoding an inducer fusion protein according to the invention comprising at least said polypeptide recognized by the binding site.

A subject of the invention is therefore also a cell, expressing a reporter gene and an effector gene, the reporter gene comprising a binding site for a polypeptide and at least one gene of interest, and the effector gene encoding an inducer fusion protein according to the invention comprising at least said polypeptide recognized by the binding site. The expression of these genes in the cell can be carried out by transfection of a reporter plasmid and of an effector plasmid, or any other means known to those skilled in the art.

Preferably, the cell is a eukaryotic cell and the polypeptide is a non-eukaryotic polypeptide, in particular a bacteriophage polypeptide, and more particularly the capsid protein of the R17 bacteriophage. In the latter case, the fusion protein will comprise the capsid protein of the R17 bacteriophage. The advantage is that the reporter gene/effector gene couple is then completely exogenous with respect to the eukaryotic system in which it is expressed, which avoids the sometimes considerable background noise associated with the unwanted induction of endogenous genes.

In a particular embodiment, the reporter gene is expressed from a bicistronic RNA comprising a first cistron and a second cistron. The intercistron space may comprise a binding site for the inducer fusion protein. Such a bicistronic structure allows great variability of reporter genes, thus multiplying the possibilities of selection, localization, labeling of expression, etc.

Many applications of the invention can be envisioned.

In particular, the invention is very advantageous in gene therapy. It is possible to complete the defective genome of an organism with a gene x, the expression of which will be precisely controlled by means of modulatable concentrations of the appropriate inhibitor. It is also possible to bring about the expression of a variable and controlled concentration of one or more genes capable of killing, or of inhibiting the proliferation of, the cells of the organism (for example, gene encoding the soluble membrane receptor fraction, gene encoding scFv antibody fragments, etc.). It is also possible to control the expression and therefore to bring about the expression of a variable concentration of anti-oncogenes (for example, p53, p70), cytokines or interleukins, growth factors, dominant negatives of certain proteins, xenogenes.

The invention can also be used for screening post-translational modification inhibitors. This screening can be carried out by means of a screening kit: kit obtained by stable integration of plasmids according to the invention into cell lines, or kit obtained by transient transfection or “naked plasmids”. A kit according to the invention can also comprise a cell according to the invention, comprising a reporter gene as described above.

A kit according to the invention makes it possible to evaluate the influence of the presence of a given agent on the post-translational modification of an inducer fusion protein, and therefore makes it possible to determine whether or not said agent is an inhibitor. By way of examples of screened inhibitors, mention may be made of antifarnesyl prenyltransferase inhibitors, antigeranyl-geranyl prenyltransferase inhibitors, palmitoylation inhibitors, myristylation inhibitors, etc.

The invention can also be used in fundamental research, for selecting the expression of certain genes in cells in culture or in laboratory animal tissues (in vitro). It is possible to generate the point or long-term induction and modulation of the expression of any proteins; rapid induction and repression of protein expression can be obtained.

Use may in particular be made of agents that are already known and are in the clinical phase for other applications (in particular inhibitors) and for which it is known that they do not have any toxic effect.

### EXAMPLE 1

The object of this example is to demonstrate the control of gene expression induction in mammalian cells in culture ex vivo.

In this example, the expression step that is induced is translation, and the post-translational modification of the inducer fusion protein is farnesylation.

Two types of plasmids will be used:

1. “Reporter” plasmids that make it possible to transcribe, in the cells, a bicistronic RNA encoding the LucR (Renilla luciferase) reporter protein in the first cistron and the LucF (firefly luciferase) reporter protein in the second cistron. The plasmids pCRL30-R17 or pCRL138-R17 also contain, in the intercistron space of the mRNA, a binding site for the capsid protein of the R17 bacteriophage (plasmid). The plasmids pCRL30 or pCRL138 (control) do not contain this site.

2. Effector plasmids which will be able to express, in the cells:
a fusion protein consisting of the R17 phage capsid protein fused with the C-terminal region of the eIF4G translation initiation factor, for the plasmid pCI R17-4G,
a fusion protein consisting of the R17 phage capsid protein fused with the C-terminal region of the eIF4G translation initiation factor and a farnesylation protein domain (of protein sequence CVLS), for the plasmid pCI R17-4G-CVLS.

The procedure is carried out in the following way. SKHep-1 human hepatoma cells are transiently transfected with cationic liposomes using 10 pmol of reporter plasmids and 5 pmol of activator plasmids per 1 million cells. 24 hours after transfection the cells are treated for various periods (1 to 8 hours) with a farnesyltransferase inhibitor, Cys-Val-3(2-naphthyl)Ala-Met; sigma C4433, at a final concentration of 15 μM in the culture medium.

After lysis of the cells, the LucR and LucF activities are assayed using the “Dual-Luciferase (TM) reporter system” kit (Promega E1980) on a Berthold LB96B luminescence device. The LucF/LucR ratio represents the translation activity of the second cistron which is under the control of the inducibility system.

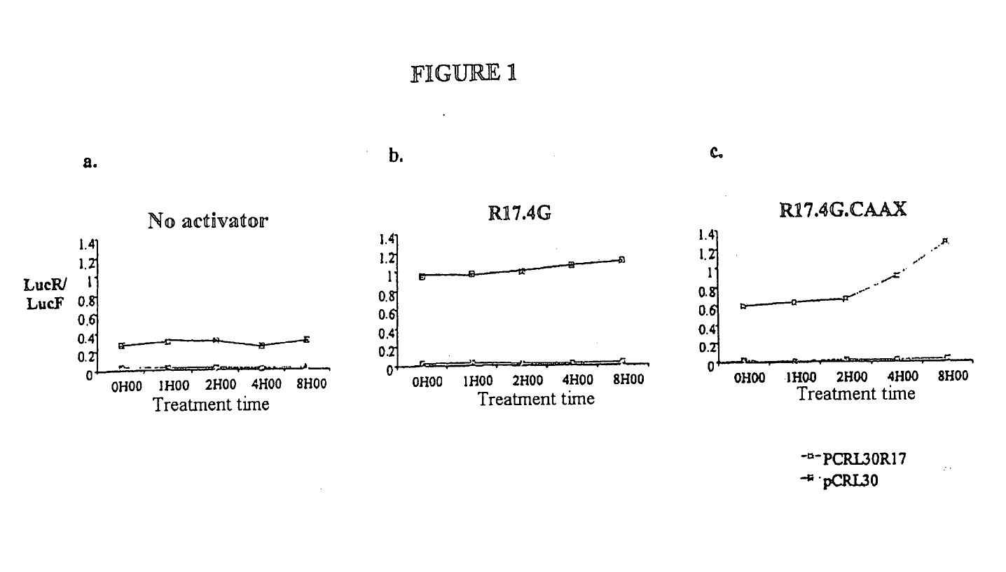

The graphs in FIG. 1 represent the change in the LucF/LucR ratio as a function of treatment time for the two types of reporter plasmid pCRL30 and pCRL30-R17. Graph a shows the change in the ratio in the absence of effector plasmid. Graph b shows the change in the ratio in the presence of plasmid expressing the fusion protein R17-4G. Graph c shows the change in the ratio in the presence of plasmid expressing the inducer fusion protein R17-4G-CVLS.

It can be seen that the ratio is low when the reporter plasmids are transfected in the absence of the effector plasmids. The translation of the second cistron is therefore low in this case (graph a). The expression of the R17-4G fusion protein allows an increase in the level of translation of the second cistron of the bicistronic reporter RNA containing the R17 site (pCRL 30R17), but not of that which does not contain the R17 site (pCRL 30) (graph b). Finally, it can be observed (graph c) that the level of translation induced by the expression of the R17-4G fusion protein is not modulated by the treatment with the farnesyltransferase inhibitor. Conversely, the increase in translation induced by the R17-4G-CAAX inducer fusion protein is modulated by using the farnesyltransferase inhibitor, and more particularly is dependent on the time of treatment with the farnesyltransferase inhibitor.

### EXAMPLE 2

The studies carried out on the SKHep cells can be extended to other cell lines, and in particular HeLa cells (derived from a uterine adenocarcinoma).

This HeLa line was used to carry out permanent integrations of effector plasmids expressing the R17-4G-CVLS fusion or of “reporter” plasmids containing (pCRL30-R17 or pCRL138-R17) or not containing (pCRL30 or pCRL138) the RNA structure recognized by the R71 protein. These permanent transfections combined with a G418 resistance gene made it possible to select cellular clones. Among the clones obtained, those which gave the best activation signal to background noise ratio were selected.

Two types of studies were carried out depending on the stable clone used.

First Study.

a) Confirmation of the Effect of the Farnesyl-Transferase Inhibitor FTI 277.

The nucleic acid encoding the R17-4G-CVLS protein is integrated into the genome of HeLa cells. The reporter plasmids pCRL138 and pCRL138-R17 are transiently transfected into this clone. The products that act on farnesylation of R17-4G-CVLS are then sought.

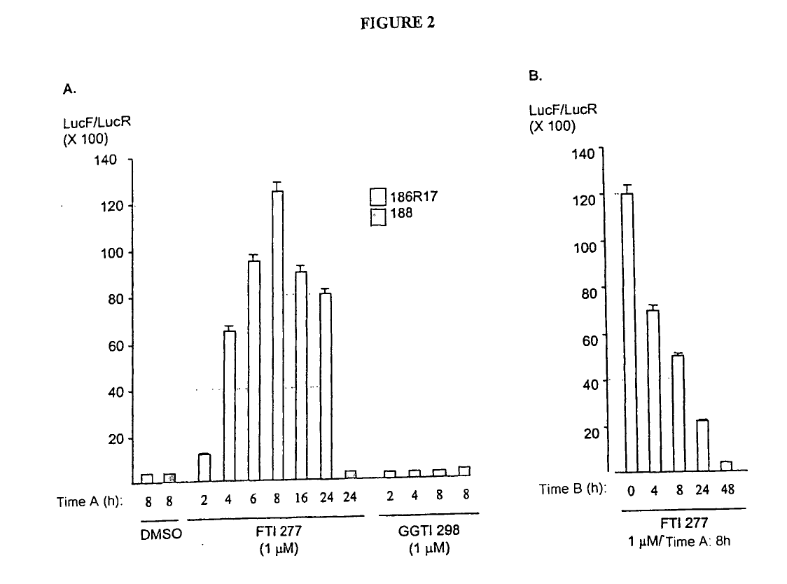

FIG. 2A confirms the effect of the farnesyltransferase inhibitor FTI 277.

DMSO is the solvent in which the products (FTI 277 and GGTI 298) that are added to the culture medium are dissolved, and therefore represents an induction control. The final concentration of DMSO in the culture medium does not exceed 0.1%.

The white rectangles represent the RNA which carries the R17 protein recognition sequence. The black rectangles represent the activities obtained under the same conditions with an RNA sequence that is similar but no longer carries the R17 recognition; it is therefore the negative control for the first construct.

The induction is represented by the quotient of the LucF and LucR values measured directly in the presence of an agent (FTI 277 or GGTI 298), relative to the DMSO control.

Thus, the kinetics of action of FTI 277 represented in FIG. 2A show that FTI 277 has an inducer effect, and that the maximum activation effect is obtained at 8 hours of treatment. It should also be noted that the treatment of the cells with a geranyl-geranyl transferase inhibitor, GGTI 298, has no activation effect, which demonstrates the specificity of the mechanism.

FIG. 2B shows that the activation effect of FTI 277 is reversible. In this experiment, the cells were treated for 8 h with 1 μM FTI 277, and then the product was removed and a rapid decrease in the LucF/LucR activation ratio was observed at the various times indicated.

b) Confirmation of the Effect of the HMGCoA-Reductase Inhibitor And Screening Of Inhibitors.

HMGCoA-reductase, which synthesizes melavonate, is involved in isoprenylation, a post-translational modification of proteins such as 4G-CVLS.

This enzyme is inhibited by statins (lovastatin, simvastatin, etc.).

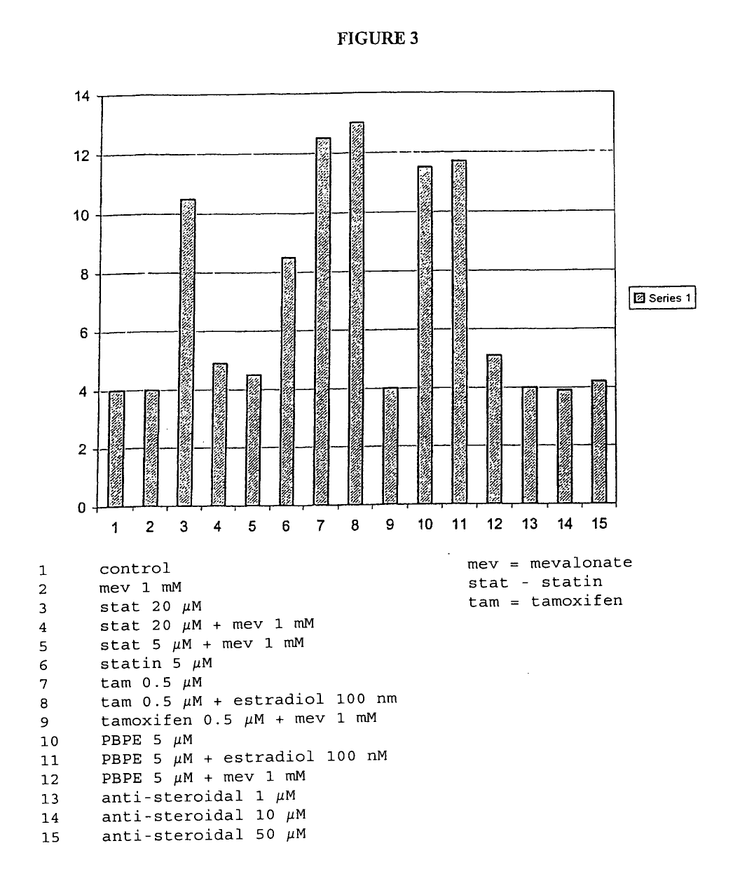

In FIG. 3, the cells used during these tests are HeLa cells stably transfected with the plasmid encoding the R17-4G-CVLS fusion protein, and transiently transfected with the bicistronic reporter plasmids pCRL138 and pCRL138-R17.

The results are given in the form of the ratio of LucF/LucR activity in the presence of various pharmacological agents in the culture medium, relative to a DMSO control.

Lines 3 and 6 in FIG. 3 show that the presence of statin inhibits HMGCoA-reductase, and therefore prenylation, and the fusion protein is not therefore targeted to the membrane and plays its activator role.

Lines 2, 4 and 5 show that the presence of mevalonate, synthesized by HMGCoA-reductase, allows, even in the presence of an HMGCoA-reductase inhibitor, isoprenylation, i.e. the post-translational modification of the fusion protein, which will therefore be targeted to the membrane and which will not be able to play an activator role, hence LucF/LucR ratios identical or similar to that of the control.

The model used also made it possible to screen potential HMGCoA-reductase inhibiting agents.

Lines 7 and 8 show that tamoxifen, an anti-estrogen, inhibits this enzyme even at low concentrations. This inhibitory activity is independent of estradiol and of the estrogen receptor (absent from the HeLa cells used) and is reversed or compensated for by mevalonate (line 9).

Other anti-estrogens were tested.

Lines 13, 14 and 15 represent the results obtained with steroidal anti-estrogens from the companies ICI and Roussel Uclaf, that have a high affinity for the estrogen receptor, at various concentrations. These anti-estrogens have no HMGCOA-reductase inhibiting activity.

On the other hand, another anti-estrogen, called PBPE, designed and produced by the inventors, which does not bind to the estrogen receptor but binds to a protein complex referred to as AEBS (anti-estrogen binding site), were found to be an inhibitor of HMGCOA-reductase (lines 10 and 11), its inhibitory activity being, as for the above inhibitors, compensated for by the presence of mevalonate.

c) Molecules that Inhibit Farnesyl Pyrophosphate Synthase, also called FPP-synthase

This enzyme is required for the biosynthesis of farnesyl pyrophosphates. Its inhibition therefore also inhibits protein prenylation.

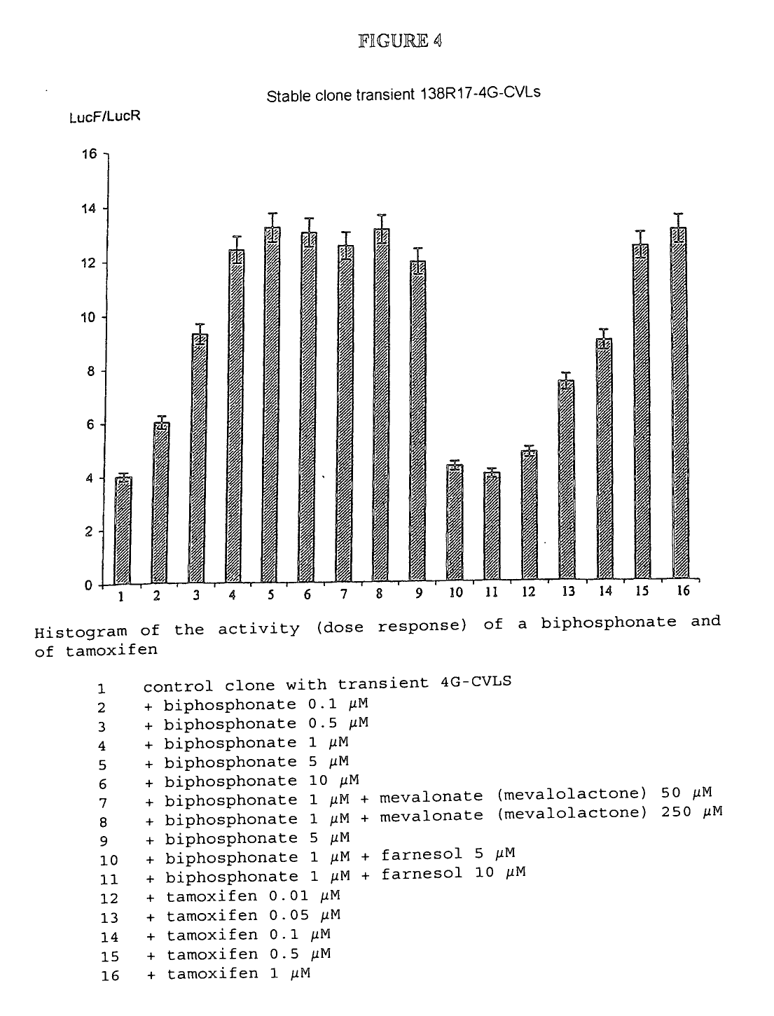

In these tests represented in FIG. 4, the same cellular model as in b), stably transfected with R17-4G-CVLS, is used.

The results of lines 2 to 6 and 9 show that biphosphonates, conventionally used as analgesics in the treatment of bone metastases, are also farnesylation inhibitors and that their presence therefore increases the LucF/LucR ratio.

The results of lines 7 and 8 show that the presence of mevalonate does not compensate for the inhibitory activity of biphosphonates. Conversely, the presence of farnesol (lines 11 and 12) reverses or compensates for the inhibitory activity of biphosphonates, which shows that this activity is not related to the inhibition of HMGCoA-reductase, but that the biphosphonates act downstream in the isoprene biosynthesis cascade, and doubtless on FPP-synthase.

The results obtained in the presence of tamoxifen (lines 12 to 16) show that tamoxifen is also an FPP-synthase inhibitor.

This model therefore made it possible to show that certain molecules unexpectedly inhibit isoprenyl biosynthesis by acting on protein prenylation. It demonstrated that two families of medicinal products (anti-estrogens and biphosphonates) that are among the most widely used in cancer therapy because they are highly innocuous, are also capable of inhibiting farnesylation.

Second Study

The pCRL138 or pCRL138-R17 reporter sequences are integrated into the genome of HeLa cells, and various R17-4G-CAAX constructs—where CAAX can be farnesylated, “CVLS”, or geranylgeranylated, “CVLL”, or no longer undergo any post-translational modifications of this type, “SVLS”,—are transiently transfected into these two clones.

After transfection, the cells are treated with FTI 277 (farnesyltransferase inhibitor) or GGTI 298 (geranyltransferase inhibitor).

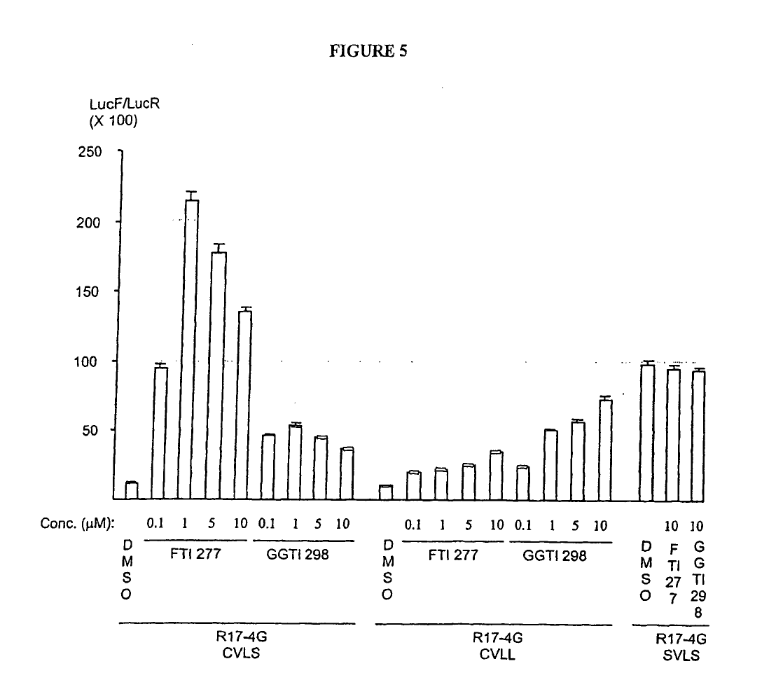

The following results, represented in FIG. 5, are observed relative to the DMSO controls:
for R17-4G-CVLS (which contains a farnesylations box), only FTI 277 is capable of activating translation,
for R17-4G-CVLL (which contains a geranylations box), only GGTI 298 is capable of activating translation,
for R17-4G-SVLS (which can be neither farnesylated or geranylated), neither of the two molecules is capable of activation.

This experiment therefore shows very good specificity of action of the inhibitors, which allows a precise control of the induction.

Moreover, the subcellular localization of proteins containing CVLS, CVLL or SVLS boxes was verified.

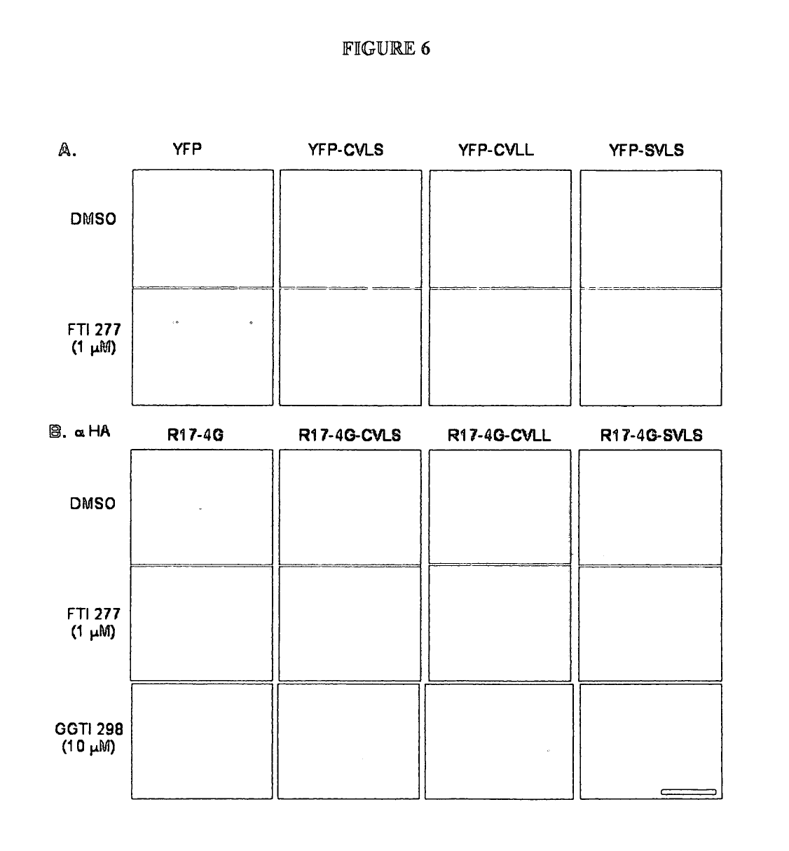

To do this, these boxes were fused with the YFP protein (yellow fluorescent protein), and all the fusion proteins were cloned with an HA sequence in the N-terminal position. The constructs were transiently transfected into HeLa cells, and the localization of the fusion proteins was followed by fluorescence microscopy after immunofluorescence with an anti-HA antibody (FIGS. 6A and B, in color).

It is noted that the fusion proteins containing CVLS or CVLL boxes have a predominantly membrane localization.

It may also be noted that the treatment with FTI 277 (8 hours) relocalizes only the YFP-CVLS and R17-4G-CVLS proteins to the cytoplasm, which is not the case of the treatment with GGTI 298.

### EXAMPLE 3

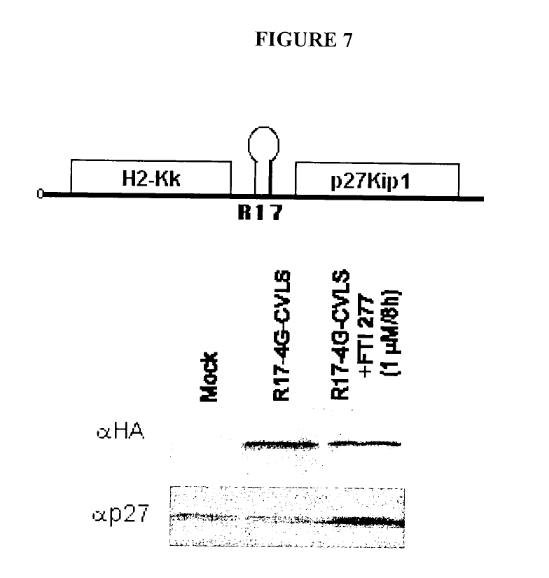

The reporter plasmid constructed in the context of this example makes it possible to transcribe, in mammalian cells in culture ex vivo, a bicistronic RNA encoding a truncated H2-Kk protein (mouse MHC class I molecule) as a marker for selection of the transfected cells, and encoding the p27Kip1 protein which is a cell cycle-inhibiting protein. The bicistronic RNA also contains, in the intercistron space, a binding site for the R17 bacteriophage capsid protein. This bicistronic RNA is very diagrammatically represented in FIG. 7.

The effector plasmid used is the one for expressing the R17-4G-CVLS fusion protein.

CHO (Chinese hamster ovary) cells are transiently transfected with cationic liposomes using 10 pmol of reporter plasmids and 5 pmol of activator plasmids per 1 million cells. Twelve hours after transfection, the cells are treated for 8 hours with the farnesyltransferase inhibitor (Cys-Val-3(2-naphthyl)Ala-Met; Sigma C4433) at a final concentration of 1 μM in the culture medium. The transfected cells are selected with the “MACSelect™ Kk” kit (Milteny Biotech). After lysis of the selected cells, the protein extracts are loaded onto an SDS-PAGE gel.

The results are given in FIG. 7. The expression of the p27 protein (ap27 line) is examined by Western blotting with a monoclonal antibody (Ref. 610241, BD Bioscience Pharmingen). The expression of the R17-4G-CVLS fusion protein (aHA line) is detected by Western blotting using a monoclonal antibody against the HA peptide (Eurogentec) located at the NH₂-terminal end of the R17-4G-CVLS protein.

The “Mock” column is the control column, which represents the expression of the p27 protein and of the fusion protein in cells not transfected with the reporter and effector plasmids. The R17-4G-CVLS column represents the expression of the p27 protein and of the fusion protein in the cells cotransfected in the absence of inhibitor. The R17-4G-CVLS+FTI277 column represents the expression of the p27 protein and of the fusion protein in the cells cotransfected after treatment with the inhibitor.

An increase in the amount of p27 is observed in the cells cotransfected with the pR17-4G-CVLS and pK-R17-p27 vectors in the presence of farnesyltransferase inhibitor. This increase is independent of the amount of fusion protein produced, identical with or without treatment with the inhibitor. On the other hand, in the presence of the inhibitor, the fusion protein is not targeted to the membrane and can play its activator role, hence the increase in the amount of p27.

1-13. (canceled)

14. A gene expression-inducing fusion protein, wherein it comprises, firstly, a ribonucleic acid-binding peptide domain and a domain for activating the post-transcriptional expression of the gene and, secondly, a domain enabling delocalization to the cytoplasmic membrane.

15. The fusion protein as claimed in claim 14, wherein the expression-activating domain is a translation-activating domain.

16. The fusion protein as claimed in claim 14, wherein the domain enabling delocalization to the cytoplasmic membrane is a farnesylation domain.

17. A nucleic acid comprising a sequence encoding a protein as claimed in claim 14.

18. An expression vector comprising a nucleic acid as claimed in claim 17.

19. A recombined cell comprising a nucleic acid as claimed in claim 17.

20. A recombined cell comprising an expression vector as claimed in claim 18.

21. A cell line of recombined cells comprising a nucleic acid as claimed in claim 17.

22. A cell line of recombined cells comprising an expression vector as claimed in claim 18.

23. A cell expressing a reporter gene and an effector gene, the reporter gene comprising a binding site for a polypeptide and at least one gene of interest, and the effector gene encoding an inducer fusion protein as claimed in claim 14 comprising at least said polypeptide recognized by the binding site.

24. The cell as claimed in claim 23, in which the cell is eukaryotic and the polypeptide is noneukaryotic.

25. The cell as claimed in claim 23, in which the reporter gene is expressed from a bicistronic RNA comprising a first cistron and a second cistron.

26. A nonhuman transgenic organism comprising a nucleic acid as claimed in claim 17.

27. A nonhuman transgenic organism comprising an expression vector as claimed in claim 18.

28. A modulatable permanent external in vitro method for controlling post-transcriptional gene expression induction in a recombined cell or in a nonhuman transgenic tissue comprising a nucleic acid comprising a sequence encoding a fusion protein as claimed in claim 14, or comprising an expression vector comprising said nucleic acid, by modulating the state of post-translational modification of the fusion protein using an appropriate inhibitor of said post-translational modification.

29. A kit for screening agents, comprising at least one cell as claimed in claim 19.
